(19) United States
(12) Plant Patent Application Publication
(10) Pub. No.: US 2009/0055978 P1
(43) Pub. Date: Feb. 26, 2009

(54) Apple tree named 'PLBAR B1'

(76) Inventor: Dennis W. Barnsby, Pemberton (AU)

Correspondence Address: STRATTON BALLEW, 213 S 12TH AVE, YAKIMA, WA, 98902, US

(21) Appl. No.: 12/214,790

(22) Filed: Jun. 20, 2008

Related U.S. Application Data
(60) Provisional application No. 60/937,325, filed on Jun. 27, 2007.

(51) Int. Cl. A01H 5/00

(52) U.S. Cl. PLT/161

(57) ABSTRACT

‘PLBAR B1’ is a new apple tree that originated as a limb sport mutation of ‘Cripps Pink.’ The new variety is notable for its exceptional color and early maturity as compared to ‘Cripps Pink.’ Fruit of the new variety is mature and ready for harvest 14 to 18 days earlier than fruit of ‘Cripps Pink.’

### CROSS-REFERENCE TO RELATED APPLICATIONS

This application claims priority of co-pending U.S. Provisional Application Ser. No. 60/937,325, filed Jun. 27, 2007.

### FEDERALLY SPONSORED RESEARCH OR DEVELOPMENT

None

### LATIN NAME OF THE GENUS AND SPECIES OF THE PLANT CLAIMED

Malus domestica

### VARIETY DENOMINATION

‘PLBAR B1’

### BACKGROUND AND SUMMARY OF THE VARIETY

‘PLBAR B1’ originated as a limb sport mutation of ‘Cripps Pink’ (U.S. Plant Pat. No. 7,880). It was discovered by the inventor in a cultivated orchard at Pemberton, Western Australia during the 1999-2000 growing season. ‘PLBAR B1’ was asexually propagated by field grafting during the 2001-2002 growing season at Pemberton, and has been shown to remain true to type over successive generations.

‘PLBAR B1’ is a new and distinct apple tree notable for its exceptional color and early maturity as compared to ‘Cripps Pink.’ Fruit of the new variety is mature and ready for harvest about 14 to 18 days earlier than fruit of ‘Cripps Pink,’ as measured by a difference of 2 to 3 points in the industry standard starch score range of 1 to 6.

### BRIEF DESCRIPTION OF THE PHOTOGRAPHS

FIG. 1 shows the fruit of ‘PLBAR B1’;

FIG. 2 shows a comparison of the fruit of ‘PLBAR B1’ (on the left) to parent variety ‘Cripps Pink’ (on the right) after starch testing.

FIGS. 3 and 4 show a tree of the ‘PLBAR B1’ variety.

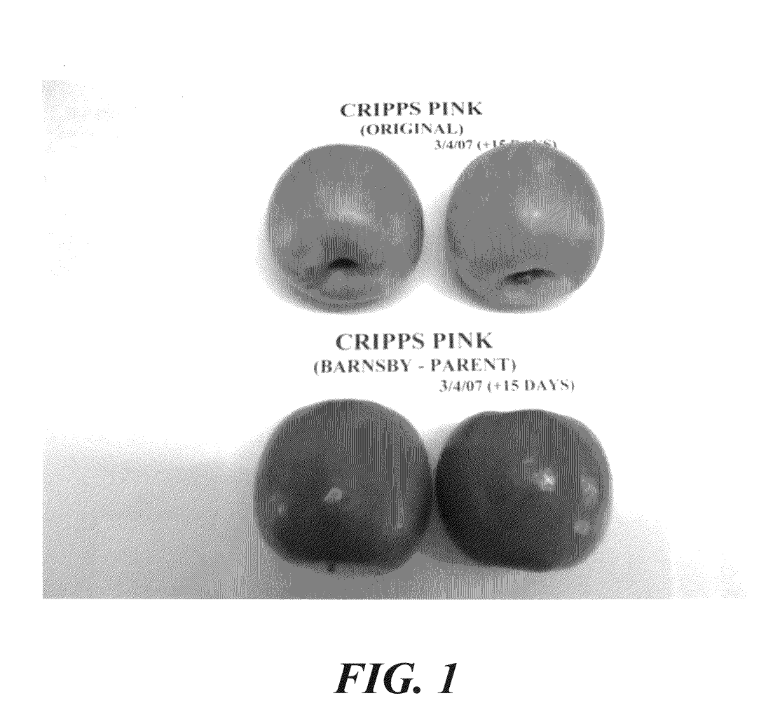

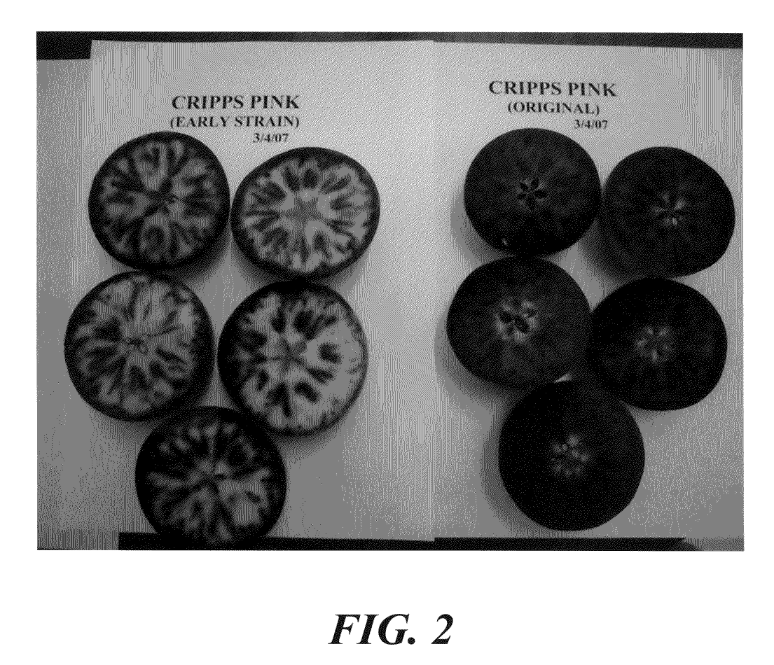

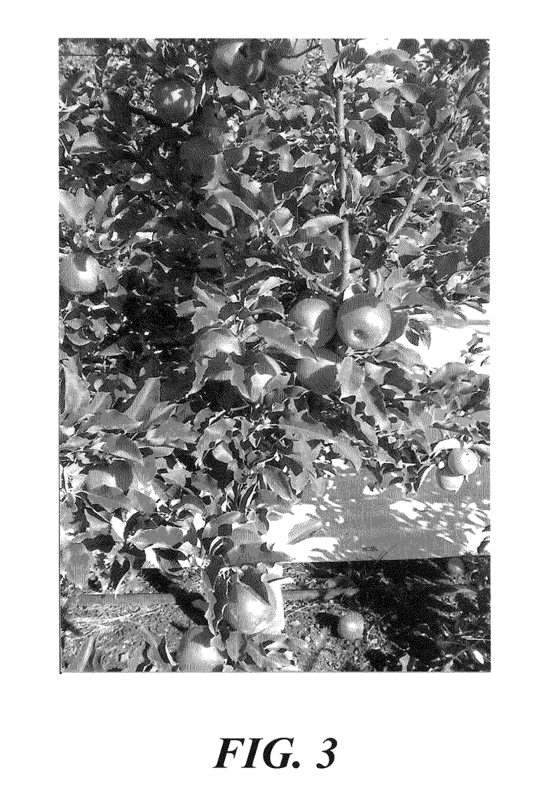

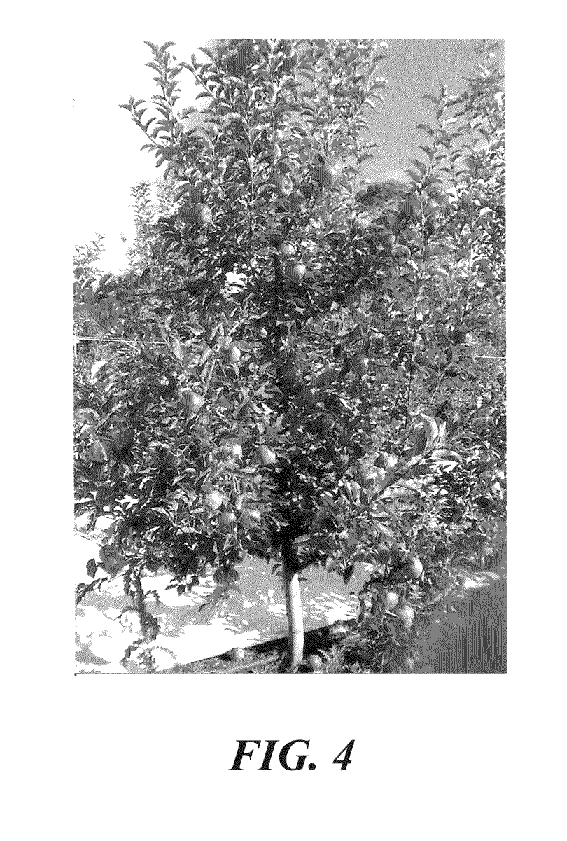

### DETAILED BOTANICAL DESCRIPTION OF THE VARIETY

The following detailed botanical description is based on observations of first generation trees planted in 2003, and described during the 2006-2007 and 2007-2008 growing seasons at Pemberton, Western Australia. Color descriptions refer to The Royal Horticultural Society Colour Chart. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Tree: Medium to large with dense foliage, upright habit; Very vigorous; Height 3.1 to 4.1 m; Spread 1.75 to 2 m.
Trunk: Medium stocky, diameter 23 cm to 25 cm at 30 cm above graft union; Bark smooth, color light brown; Lenticels — medium density, cream in color, typical of ‘Cripps Pink.’
Branches: Thick, smooth, upright; Branching habit — Much branched with average branching angles (inside measurement) of 45 degrees to 50 degrees if allowed to grow naturally; Color — Green-brown; Shape of tipbud — Rounded; Lenticels — medium low density, cream in color.
Winter hardiness: Equivalent to ‘Cripps Pink.’
Chilling requirement: 400 to 500 hours below 7° C.
Dormant one year old shoot: Pubescence medium; Size — diameter 4 to 6 mm; Color medium brown; Internode length 49 to 56 mm.
Bloom period: Late; First bloom October 24 at Pemberton; Full bloom November 5 at Pemberton.
Leaves: Large, wide, long, oval, convex, pointed, medium thick, smooth; Length — 84 to 111 mm; Width — 45 to 56 mm; Color — Green, medium glossy on upper surface, green with weak pubescence on lower surface; Margin — Finely serrate, crenate; Petiole — Long, 26 to 41 mm, medium slender, pubescent. Color — Light green; Stipule — Small; Central leaf vein — Color light green with pink tinge toward basal end.
Fruit: Maturity when described — Eating ripe. Size — Large, uniform; Length — 73 mm; Breadth — 78 mm; Mean fruit weight — 160 to 195 g; Form — Asymmetrical, ellipsoid-conical with a slight irregular surface; Medium distal crowns, rounded at base, sides slightly unbroken, unequal; Axis — Nonvertical; Cavity — Acute, deep 21 mm, medium width 34 mm, symmetrical, greenish, with very slight unbroken russet; Basin — Medium crown, ribbed, wide 32 mm, open, medium depth 12 mm; Markings — None; Sepals — Medium, touching; Stem — Medium length 22 mm, medium thickness 3 mm, not lipped; Calyx — Closed, V-shaped, medium width and medium depth; Calyx lobes — Reflexed and divergent; Pubescence — None.
Skin: Texture — Slightly bumpy, greasy; Bloom — absent; Cracking tendency — absent; Thickness — medium; Ground color — yellow-green; Overcolor — pink-red; Percentage of overcolor — 70% to 80%; Russet — none. Lenticels — medium, numerous, roundish.
Flesh: Juicy, firm; Color — Créme; Texture — Firm; Flavor — Rich, complex, sub acid to sweet; Aroma — Distinct, complex and highly aromatic; Browning of the flesh (one hour after being cut, with stainless steel knife) — nil; Quality — Best.
Core: Median. Bundle area — Medium small, symmetrical. Halves of area — Equal. Bundles — Inconspicuous. Core lines — Meeting, heart-shaped. Calyx tube — Funnel-form. Pubescence — none. Stem or funnel — Medium long. Depth of tube to shoulder — About 5 mm. Entire depth — About 12 mm. Styles — Some present, united at base. Stamens — Median, in one whorl. Carpels — Closed, axile, symmetrical, smooth, cordate form, emarginate at outer edge near tip.
Seeds: Quantity — One or two per cell, not tufted, acute at point, 8-9 mm long, 5 mm wide, obtuse, dark brown.
Harvest Date: About 14 to 18 days before ‘Cripps Pink.’
Resistance to known diseases: None noted
Keeping quality: Good.

I claim:

1. A new and distinct apple tree substantially as described and illustrated herein.
